(19) United States
(12) Patent Application Publication
(10) Pub. No.: US 2004/0014687 A1
(43) Pub. Date: Jan. 22, 2004

(54) Macrolide antibacterial compounds

(76) Inventors: Zhenkun Ma, Dallas, TX (US); Stevan Djuric, Libertyville, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/420,389

(22) Filed: Apr. 22, 2003

Related U.S. Application Data
(60) Provisional application No. 60/375,513, filed on Apr. 25, 2002.

(51) Int. Cl. C07H 17/08; A61K 31/7048

(52) U.S. Cl. 514/28; 536/7.1

(57) ABSTRACT

Macrolide antibacterial compounds having formula (I)

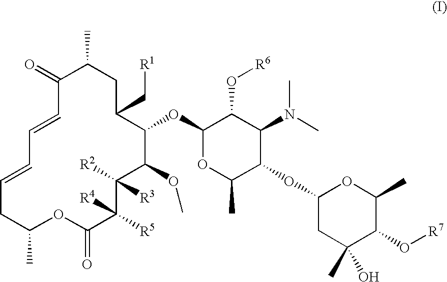

and formula (II)

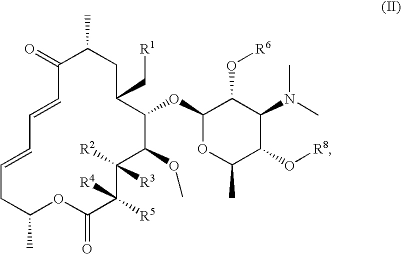

and salts, prodrugs, and salts of prodrugs thereof, processes for making the compounds and intermediates employed in the processes, compositions containing the compounds, and methods for prophylaxis or treatment of bacterial infections in a fish or a mammal using the compounds are disclosed.

This application claims benefit of co-pending U.S. Application Serial No. 60/375,513, filed Apr. 25, 2002, the specification of which is hereby incorporated by reference into this application.

### TECHNICAL FIELD

This invention is directed to compounds which are useful as antibacterials, processes for making the compounds and intermediates used in the processes, compositions containing the compounds, and methods for prophylaxis or treatment of bacterial infections using the compounds.

### BACKGROUND OF THIS INVENTION

Because the effectiveness of many drugs currently available for prophylaxis or treatment of bacterial infections is being compromised by the emergence of drug-resistant bacteria, novel antibacterials would be beneficial for their therapeutic value and their contribution to the antibacterial arts.

### SUMMARY OF THE INVENTION

A first embodiment of this invention, therefore, is directed to compounds, and salts, prodrugs, and salts of prodrugs thereof, which are useful as antibacterials, the compounds having formula (I)

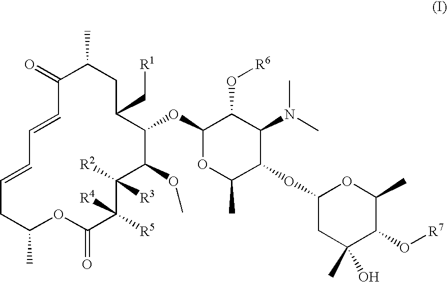

and formula (II)

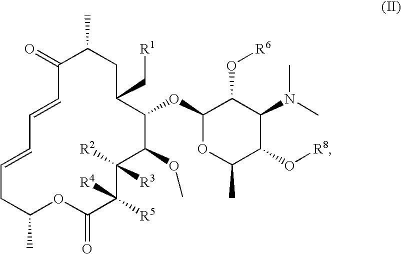

in which

R¹ is hydrogen, alkyl, cycloalkyl, alkenyl, alkynyl, —R⁹, C(O)H, —C(O)OH, —C(O)R¹⁰, —C(O)NR¹¹R¹², —C(H)═N—OR¹³ —C(H)═NR¹⁴, —CH₂N (H) R¹⁵, or —CH₂NR¹⁵R¹⁶;

one of R²or R³is hydrogen and the other is —OH, —OCH₂R⁹, OC(O)R¹⁰, —OC(O)NR¹¹R¹², —OCH₂C(H)═N—OR¹³, or —OCH₂C(H)═NR¹⁴; or

R² and R³ together are ═O;

one of R⁴ or R⁵ is hydrogen and the other is hydrogen or fluoro;

R⁶, R⁷, and R⁸ are independently hydrogen, alkyl, cycloalkyl, —(CH₂)alkenyl, —(CH₂)alkynyl, —R^{P}, —CH₂R⁹, —C(O)R¹⁰, —C(O)NR¹¹R¹², —(CH₂)C(H)═N—OR¹³, —(CH₂)₂C(H)═NR¹⁴, —(CH₂)₂N(H)R¹⁵, —(CH₂)₂NR¹⁵R¹⁶, alkyl substituted with one substituent selected from the group consisting of aryl, heteroaryl, and heterocyclyl, —(CH₂)alkenyl substituted with one substituent selected from the group consisting of —NR¹⁵R¹⁶, aryl, heteroaryl, and heterocyclyl, or —(CH₂)alkynyl substituted with one substituent selected from the group consisting of —NR¹⁵R¹⁶, aryl, heteroaryl, and heterocyclyl;

R^{P} is a hydroxyl protecting moiety;

R⁹ is alkyl interrupted with one or two moieties independently selected from the group consisting of —O—, —S—, —S(O)—, and —SO₂—, alkenyl interrupted with one moiety selected from the group consisting of —O—, —S—, —S(O)—, and —SO₂—, or alkynyl interrupted with one moiety selected from the group consisting of —O—, —S—, —S(O)—, and —SO₂—,

in which each R⁹ moiety is unsubstituted or substituted with one, two, or three substituents independently selected from the group consisting of cycloalkyl, halo, aryl, heteroaryl, heterocyclyl, —OH, ═O, —NH₂, —NH(alkyl), and —N(alkyl)₂;

R¹⁰ is alkyl, alkenyl, alkynyl, cycloalkyl, —R¹⁷, alkyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, alkenyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, or alkynyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂;

R¹¹, R¹², R¹³, R¹⁴, and R¹⁵ are independently alkyl, —(CH₂)alkenyl, —(CH₂)alkynyl, cycloalkyl, —CH₂R¹⁷, alkyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, alkenyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, or alkynyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂;

R¹⁶ is hydrogen or R¹⁵; and

R¹⁷ is alkyl interrupted with one or two moieties independently selected from the group consisting of —O—, —S—, —S(O)—, and —SO₂—, alkenyl interrupted with one moiety selected from the group consisting of —O—, —S—, —S(O)—, and —SO₂—, or alkynyl interrupted with one moiety selected from the group consisting of —O—, —S—, —S(O)—, and —SO₂—,

in which each R¹⁷ moiety is unsubstituted or substituted with one, two, or three substituents independently selected from the group consisting of cycloalkyl, halo, aryl, heteroaryl, heterocyclyl, —OH, ═O, —NH₂, —NH(alkyl), and —N(alkyl)₂.

A second embodiment of this invention is directed to a process for making the compounds.

A third embodiment of this invention is directed to intermediates which are employed in the second embodiment.

A fourth embodiment this invention is directed to compositions which are useful for the prophylaxis or treatment of bacterial infections in a fish or a mammal, the compositions comprising a therapeutically effective amount of one or more of the compounds of the first embodiment and an excipient.

A fifth embodiment of this invention is directed to methods for prophylaxis or treatment of bacterial infections in a fish or a mammal comprising administering to the fish or the mammal a therapeutically effective amount of one or more of the compounds of the first embodiment.

### DETAILED DESCRIPTION OF THE INVENTION

Compounds of this invention, also referred to as “the compounds,” comprise both fixed and variable components or “moieties,” which variable moieties are identified by a capital letter and accompanying numerical or alphabetical superscript, and for which the following terms have the meanings indicated.

“Alkenyl” means monovalent, straight-chain and branched-chain hydrocarbon moieties, having two to eight carbon atoms and at least one carbon-carbon double bond.

Alkenyl moieties include but-1,3-dienyl, butenyl, but-2-enyl, ethenyl, 1-ethylhexen-2-yl, hex-3-enyl, 1-methylbutenyl, 2-methylbutenyl, 1-methylbut-2-enyl, 1-methylbut-1,3-dienyl, pentenyl, pent-2-enyl, pent-3-enyl, and propenyl.

“Alkyl” means monovalent, saturated, straight-chain and branched-chain hydrocarbon moieties, having one to six carbon atoms.

Alkyl moieties include butyl, 1,1,-dimethylethyl, 1,1-dimethylpropyl, 1,2-dimethylpropyl, ethyl, 1-ethylpropyl, 2-ethylpropyl, hexyl, methyl, 2-methylpropyl, 3-methylbutyl, 1-methylpentyl, 2-methylpent-3-yl, and pentyl.

“Alkylene” means divalent, saturated, straight-chain and branched-chain hydrocarbon moieties, having one to eight carbon atoms.

Alkylene moieties include butylene, 1,1,-dimethylethylene, 1,1-dimethylpropylene, 1,2-dimethylpropylene, ethylene, 1-ethylpropylene, 2-ethylpropylene, hexylene, methylene, 2-methylpropylene, 3-methylbutylene, 1-methylpentylene, 2-methylpent-3-ylene, and pentylene.

“Alkynyl” means monovalent, straight-chain and branched-chain hydrocarbon moieties, having two to six carbon atoms and at least one carbon-carbon triple bond.

Alkynyl moieties include ethynyl (acetylenyl), pentynyl, pent-2-ynyl, pent-3-ynyl, pent-4-ynyl, 1-methylbut-2-ynyl, 2-methylbut-3-ynyl, hexynyl, hex-2-ynyl, hex-3-ynyl, hex-4-ynyl, 1-methyl-pent-2-ynyl, 1-methylenepent-3-ynyl, 1-methyl-pent-2,4-diynyl, and prop-2-ynyl (propargyl).

“Aryl” means monovalent, unsubstituted and substituted phenyl moieties, attached through a carbon atom, and unfused or fused with another phenyl moiety or a cycloalkyl, cycloalkenyl, heteroaryl, heterocyclyl, naphthyl, or saturated part of an indanyl moiety.

Phenyl moieties fused with phenyl, naphthyl, or the saturated part of an indanyl moieties are unsubstituted and substituted naphthyl, anthracen-(1- to 4-)yl, or fluoren-(1- to 4-)yl, respectively.

Phenyl moieties fused with cycloalkyl moieties are unsubstituted and substituted indan-(4- to 7-)yl and 1,2,3,4-tetrahydronaphth-(5- to 8-)yl.

Phenyl moieties fused with cycloalkenyl moieties are unsubstituted and substituted inden-(4- to 7-)yl, 1,2-dihydronaphth-(5- to 8-)yl and 1,2-dihydronaphth-(5- to 8-)yl.

Phenyl moieties fused with heteroaryl moieties include unsubstituted and substituted benzimidazol-(4- to 7-)yl, 1-benzofuran-(4- to 7-)yl, 1,2-benzisothiazol-(4- to 7-)yl, benzthiazol-(4- to 7-)yl, 1-benzothiophen-(4- to 7-)yl, cinnolin-(5- to 8-)yl, indol-(4- to 7-)yl, isoquinolin-(5- to 8-)yl, phthalazin-(5- to 8-)yl, quinazolin-(5- to 8-)yl, quinolin-(5- to 8-)yl, and quinoxalin-(5- to 8-)yl.

Phenyl moieties fused with heterocyclyl moieties include unsubstituted and substituted 1,3-benzodiox-(4- to 7-)yl, 1,4-benzodiox-(5- to 8-)yl, 1,3-dihydro-2-benzofuran-(4- to 7-)yl, 2,3-dihydro-l-benzofuran-(4- to 7-)yl, 1,3-dihydro-2-benzothiophen-(4- to 7-)yl, 2,3-dihydro-l-benzothiophen-(4- to 7-)yl, and indolin-(4- to 7-)yl.

“Cycloalkyl” means monovalent, unsubstituted and substituted, saturated cyclic hydrocarbon moieties, having three to six carbon atoms.

Cycloalkyl moieties are unsubstituted and substituted cyclopropyl, cyclobutyl, cyclopentyl, and cyclohexyl.

“Cycloalkenyl” means means monovalent, unsubstituted and substituted, cyclic hydrocarbon moieties having four to six carbon atoms and at least one carbon-carbon double bond.

Cycloalkenyl moieties are unsubstituted and substituted 1,3-cyclohexadienyl, 1,4-cyclohexadienyl, cyclohexenyl, cyclopentadienyl, and cyclopentenyl.

“Halo” means fluoro (—F), chloro (—Cl), bromo (—Br), and iodo (—I) moieties.

“Heteroaryl” means monovalent, aromatic, unsubstituted and substituted five-membered ring moieties having two double bonds and (a) one oxygen or one sulfur atom, (b) one, two, three, or four nitrogen atoms, or (c) one or two nitrogen atoms and one oxygen or one sulfur atom and the remaining atoms are carbon atoms, each of which is attached through a carbon atom or a nitrogen atom; and monovalent six-membered ring moieties having three double bonds and one, two, or three nitrogen atoms and the remaining atoms are carbon atoms, attached through a carbon atom; in which the foregoing heteroaryl moieties are unfused or fused with another heteroaryl moiety or an aryl moiety.

Five-membered heteroaryl moieties are unsubstituted and substituted furanyl, imidazolyl, isothiazolyl, isoxazolyl, 1,2,3-oxadiazolyl, oxazolyl, pyrazolyl, pyrrolyl, tetrazolyl, 1,3,4-thiadiazolyl, thiazolyl, thiophenyl (thienyl), 2H-tetraazolyl, and 1,2,3-triazolyl.

Five-membered heteroaryl moieties fused with aryl moieties include unsubstituted and substituted benzimidazol-(1- or 2-)yl, 1-benzofuran-(2- to 3-)yl, 1,2-benzisothiazol-3-yl, benzthiazol-2-yl, 1-benzothiophen-(2- to 3-)yl, cinnolin-(3- or 4-)yl, indol-(1- to 3-)yl, isoquinolin-(1-, 3-, or 4-)yl, phthalazin-(1- or 4-)yl, quinazolin-(2- or 4-)yl, quinolin-(2- to 4-)yl, and quinoxalin-(2- or 3-)yl.

Five-membered heteroaryl moieties fused with other five-membered heteroaryl moieties include unsubstituted and substituted [1,3]thiazoio[4,5-d][1,3]oxazolyl, [1,3]thiazolo[4,5-d][1,3]thiazolyl, thieno[3,2-d][1,3]oxazolyl, thieno[3,2-d][1,3]thiazolyl, and thieno[2,3-b]thiophenyl.

Five-membered heteroaryl moieties fused with six-membered heteroaryl moieties include unsubstituted and substituted furo[2,3-b]pyridin-(2- or 3-)yl, 3H-imidazo[4,5-b]pyridin-(2- or 3-)yl, [1,3]thiazolo[4,5-b]pyrazin-2-yl, [1,3]thiazolo[4,5-b]pyridin-2-yl, and thieno[2,3-b]pyridin-(2- or 3-)yl.

Six-membered heteroaryl moieties are unsubstituted and substituted pyrazinyl, pyridazinyl, pyridyl, pyrimidinyl, and 1,3,5-triazinyl.

Six-membered heteroaryl moieties fused with aryl moieties include unsubstituted and substituted cinnolin-(3- or 4-)yl, isoquinolin-(1-, 3-, or 4-)yl, phthalazin-(1- or 4-)yl, quinazolin-(2- or 4-)yl, quinolin-(2- to 4-)yl, and quinoxalin-(2- or 3-)yl.

Six-membered heteroaryl moieties fused with five-membered heteroaryl moieties include unsubstituted and substituted furo[2,3-b]pyridin-(4- to 6-)yl, 3H-imidazo[4,5-b]pyridin-(5- to 7-)yl, [1,3]thiazolo[4,5-b]pyrazin-(5- or 6-)yl, [1,3]thiazolo[4,5-b]pyridin-(5- to 7-)yl, and thieno[2,3-b]pyridin-(4- to 6-)yl.

Six-membered heteroaryl moieties fused with other six-membered heteroaryl moieties include unsubstituted and substituted 1,5-naphthyridinyl, 1,7-naphthyridinyl, 1,8-naphthyridinyl, pteridinyl, pyridazino[4,5-d]pyridazinyl, pyrido[2,3-d]pyridazinyl, and pyrido[3,4-d]pyridazinyl.

“Heterocyclyl” means (a) monovalent, non-aromatic, unsubstituted and substituted four-membered ring moieties having one nitrogen, oxygen, or sulfur atom and the remaining atoms are carbon atoms, zero double bonds, attached through a carbon atom or a nitrogen atom, (b) monovalent, non-aromatic, unsubstituted and substituted five-membered ring moieties having one or two nitrogen, oxygen, or sulfur atoms and the remaining atoms are carbon atoms, and zero or one double bonds, attached through a carbon atom or a nitrogen atom, and (c) monovalent, non-aromatic, unsubstituted and substituted six-membered ring moieties having one, two, or three nitrogen, oxygen, or sulfur atoms and the remaining atoms are carbon atoms, and zero, one, or two double bonds, attached through a carbon atom or a nitrogen atom.

Four-membered heterocyclyl moieties are unsubstituted and substituted oxetane, thietane, and azetidine.

Five-membered heterocyclyl moieties include unsubstituted and substituted 1,4-dioxanyl, 1,3-dioxolanyl, imidazolidinyl, 2-imidazolinyl, 4,5-dihydroisoxazolyl, pyrazolidinyl, 2-pyrazolinyl, pyrrolidinyl, 2-pyrrolinyl, 3-pyrrolinyl, and 2H-pyrrolyl.

Six-membered heterocyclyl moieties include unsubstituted and substituted 1,3-dithianyl, 1,4-dithianyl, morpholinyl, piperidinyl, piperazinyl, pyranyl, 2H-pyranyl, 4H-pyranyl, and thiomorpholinyl.

Substituted aryl and heteroaryl moieties are those moieties substituted with one, two, three, four, or five substituents independently selected from the group consisting of alkyl, alkenyl, alkynyl, cycloalkyl, halo, —CN, —OH, —SH, —NH₂, —NO₂, —CF₃, —CH₂CF₃, —CF₂CF₃, —OCF₃, —OCH₂CF₃, —OCF₂CF₃, —OR³⁰, —SR³⁰, —S(O)(alkyl), —SO₂(alkyl), —C(O)H, —C(O)(alkyl), —C(O)OH, —C(O)O(alkyl), —NH(alkyl), —N(alkyl)₂, —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), —NHC(O)N(alkyl) 2, —SO₂NH₂, —SO₂NH(alkyl), —SO₂N(alkyl)₂, and R⁴⁰, in which R³⁰ is alkyl or alkyl substituted with one substituent selected from the group consisting of halo, —O(alkyl), and —S(alkyl), and R⁴⁰ is furyl, imidazolyl, indazolidinyl, isoquinolyl, isothiazolyl, isoxazolyl, morpholinyl, naphthyl, naphthyridyl, 1,2,3-oxadiazolyl, oxazolyl, phenyl, piperidinyl, piperazinyl, pyrazinyl, pyrazolyl, pyridyl, pyrimidinyl, pyrrolidinyl, pyrrolyl, quinazolyl, quinolyl, quinoxalyl, tetrazolyl, 1,2,3-thiadiazolyl, 1,3,4-thiadiazolyl, thiazolyl, thienyl, 1,2,3-triazolyl, or thiomorpholinyl, in which each R⁴⁰ moiety is unsubstituted or substituted with one, two, or three substituents independently selected from the group consisting of alkyl, alkenyl, alkynyl, cycloalkyl, halo, ═O, —CN, —OH, —SH, —NO₂, —CF₃, —CH₂CF₃, —CF₂CF₃, —OCF₃, —OCH₂CF₃, —OCF₂CF₃, —O(alkyl), —S(alkyl), —S(O) (alkyl), —SO₂(alkyl), —C(O)H, —C(O) (alkyl), —C(O)OH, —C(O)O(alkyl), —NH₂, —NH(alkyl), —N(alkyl)₂, —C(O)NH₂, —C(O)NH(alkyl), —C(O)N(alkyl)₂, —OC(O) (alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), —NHC(O)N(alkyl)₂, —SO₂NH₂, —SO₂NH(alkyl), and —SO₂N(alkyl)₂.

Substituted cycloalkyl, cycloalkenyl, and heterocyclyl moieties are those moieties substituted with one, two, or three substituents independently selected from the group consisting of alkyl, phenyl, halo, —CN, —OH, —NH₂, —CF₃, —PR³⁰, —SR³⁰, —S(O)(alkyl), —SO₂(alkyl), —C(O)H, —C(O)(alkyl), —C(O)OH, —C(O)O(alkyl), —NH(alkyl), —N(alkyl)₂, —C(O)NH₂, —C(O)NH(alkyl), and —C(O)N(alkyl)₂, in which the phenyl is unsubstituted or substituted with one, two, or three substituents independently selected from the group consisting of halo, —CN, —OH, —NH₂, and —CF₃.

“Hydroxyl protecting moiety” means selectively introducible and removable moieties which protect —OH moieties against undesirable side reactions. Hydroxyl protecting moieties include 4-nitrobenzyloxycarbonyl, 4-bromobenzyloxycarbonyl, 4-methoxybenzyloxycarbonyl, 3,4-dimethoxybenzyloxycarbonyl, tert-butoxycarbonyl, diphenylmethoxycarbonyl, 2,2,2-trichloroethoxycarbonyl, 2,2,2-tribromoethoxycarbonyl, 2-(trimethylsilyl)ethoxycarbonyl, 2-(phenylsulfonyl)ethoxycarbonyl, allyloxycarbonyl, acetyl, chloroacetyl, dichloroacetyl, trichloroacetyl, trifluoroacetyl, methoxyacetyl, phenoxyacetyl, pivaloyl, propionyl, 2-methylpropionyl, benzoyl, tert-butyl, 2,2,2-trichloroethyl, 2-trimethylsilylethyl, 1,1-dimethyl-2-propenyl, 3-methyl-3-butenyl, para-methoxybenzyl, 3,4-dimethoxybenzyl, diphenylmethyl, triphenylmethyl, tetrahydrofuryl, benzyloxymethyl, 2-methoxyethoxymethyl, 2,2,2-trichloroethoxymethyl, 2-(trimethylsilyl)ethoxymethyl, methanesulfonyl, para-toluenesulfonyl, trimethylsilyl, triethylsilyl, triisopropylsilyl, diethylisopropylsilyl, tert-butyldimethylsilyl, tert-butyldiphenylsilyl, diphenylmethylsilyl, and tert-butylmethoxyphenylsilyl.

These variable moieties may combine to provide a sixth embodiment of this invention, which embodiment is directed to compounds having formula (I) and formula (II), and salts, prodrugs, and salts of prodrugs thereof, in which

R¹ is hydrogen, alkyl, cycloalkyl, alkenyl, alkynyl, —C(O)H, —C(O)OH, —C(O)R¹⁰, —C(O)NR¹¹R¹², —C(H)═N—OR¹³, —C(H)═NR¹⁴, —CH₂N(H)R¹⁵, or —CH₂NR¹⁵R¹⁶;

one of R² or R³ is hydrogen and the other is —OH; or

R² and R³ together are ═O;

one of R⁴ or R⁵ is hydrogen and the other is hydrogen or fluoro;

R⁶ is hydrogen or —R^{P};

R⁷ and R⁸ are independently hydrogen, —R^{P} or —C(O)R¹⁰;

R^{P} is a hydroxyl protecting moiety;

R¹⁰ is alkyl, alkenyl, alkynyl, cycloalkyl, alkyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, alkenyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, or alkynyl substituted with one, two, or three substituents independently selected from the group consisting of cycloalkyl, halo, aryl, heteroaryl, heterocyclyl, —OH, ═O, —NH₂, —NH(alkyl), and —N(alkyl) ₂;

R¹¹, R¹², R¹³, R¹⁴, and R¹⁵ are independently alkyl, —(CH₂)alkenyl, —(CH₂)alkynyl, cycloalkyl, alkyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, alkenyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, or alkynyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂; and

R¹⁶ is hydrogen or R ¹⁵;

compounds having formula (I) and formula (II), and salts, prodrugs, and salts of prodrugs thereof, in which

R¹ is —C(H)═NR¹⁴, —CH₂N(H)R¹⁵, or —CH₂NR¹⁵R¹⁶;

one of R² or R³ is hydrogen and the other is —OH; or

R² and R³ together are ═O;

one of R⁴ or R⁵ is hydrogen and the other is hydrogen or fluoro;

R⁶ is hydrogen or —R^{P};

R⁷, and R⁸ are independently hydrogen, —R^{P} or —C(O)(alkyl);

R^{P} is a hydroxyl protecting moiety; and

R¹⁴ and R¹⁵ are independently alkyl, —(CH₂)alkenyl, —(CH₂)alkynyl, cycloalkyl, alkyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, alkenyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, or alkynyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂; and

R ¹⁶is hydrogen or R¹⁵;

compounds of formula (I) and formula (II), and salts, prodrugs, and salts of prodrugs thereof, in which

R¹ is —C(H)═NR¹⁴, —CH₂N(H)R¹⁵, or —CH₂NR¹⁵R¹⁶;

one of R² or R³ is hydrogen and the other is —OH; or

R² and R³ together are ═O;

one of R⁴ or R⁵ is hydrogen and the other is hydrogen or fluoro;

R⁶ is hydrogen or —R^{P};

R⁷ and R⁸ are independently hydrogen, —R^{P} or —C(O) (alkyl);

R^{P} is a hydroxyl protecting moiety;

R¹⁴ and R¹⁵ are independently alkyl, —(CH₂)alkenyl, —(CH₂)alkynyl, cycloalkyl, or alkyl substituted with one substituent selected from the group consisting of phenyl, pyridyl, pyridmidyl, thienyl, imidazolyl, oxazolyl, isoxazolyl, thiazolyl, oxadiazolyl, thiadiazolyl and triazolyl, each of which is independently unfused or fused with a moiety selected from the group consisting of phenyl, pyridyl, and pyrimidyl and independently unsubstituted or substituted with one substituent selected from the group selected from phenyl, pyridyl, pyridmidyl, thienyl, imidazolyl, oxazolyl, isoxazolyl, thiazolyl, oxadiazolyl, thiadiazolyl and triazolyl; and

R¹⁶ is hydrogen or R¹⁵; and

compounds having formula (I) and formula (II), and salts, prodrugs, and salts of prodrugs thereof, in which

R¹ is (prop-2-enyl)aminomethyl, (prop-2-ynyl)aminomethyl, (((phenyl)methyl)amino)methyl, (quinolin-3-ylmethyl)aminomethyl, (isoquinolin-3-ylmethyl)aminomethyl, ((2-(phenyl)ethyl)amino)methyl, ((3-(phenyl)propyl)aminomethyl, ((pyridin-3-yl)methyl)aminomethyl; ((5-pyridin-2-yl)thien-2-yl)methyl)aminomethyl, (((3-pyridin-2-yl)isoxazol-5-yl)methyl)aminomethyl, (((5-pyrimidin-2-yl)thien-2-yl)methyl)aminomethyl, or (((4-(1,2,3-thiadiazol-5-yl)phenyl)methyl)amino)methyl;

one of R² or R³ is hydrogen and the other is —OH; or

R² and R³ together are ═O;

one of R or R5 is hydrogen and the other is hydrogen or fluoro;

R⁶ is hydrogen; and

R⁷ and R⁸ are independently hydrogen, —C(O)(methyl), —C(O)(ethyl), —C(O)(propyl), or —C(O)((2-methyl)propyl).

Specific examples of R¹ moieties for the practice of this invention using compounds having formula (I) are ((2-(phenyl)ethyl)amino)methyl, (((phenyl),methyl)amino)methyl, ((3-(phenyl)propyl)amino)methyl, ((pyridin-3-yl)methyl)aminomethyl, and (((4-(1,2,3-thiadiazol-5-yl)phenyl)methyl)amino)methyl.

A specific example of an R¹ moiety for the practice of this invention using compounds having formula (II) is (((4-(1,2,3-thiadiazol-5-yl)phenyl)methyl)amino)methyl.

A specific example of an R² moiety for the practice of this invention using compounds having formula (I) or formula (II) is —OH.

A specific example of an R³ moiety for the practice of this invention using compounds having formula (I) or formula (II) is hydrogen.

A specific example of an R⁴ moiety for the practice of this invention using compounds having formula (I) or formula (II) is hydrogen.

A specific example of an R⁵ moiety for the practice of this invention using compounds having formula (I) or formula (II) is hydrogen.

A specific example of an R⁶ moiety for the practice of this invention using compounds having formula (I) or formula (II) is hydrogen.

Specific examples of an R⁷ moiety for the practice of this invention using compounds having formula (I) are hydrogen and —C(O)((2-methyl)propyl).

A specific example of an R⁸ moiety for the practice of this invention using compounds having formula (II) is hydrogen.

These specific moieties of the compounds may combine with the fixed moieties thereof to form a seventh embodiment of this invention, which embodiment is directed to compounds, and salts, prodrugs, and salts of prodrugs thereof, which are useful as antibacterials, the compounds having formula (I)

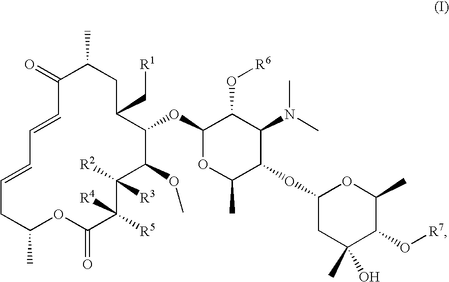

in which R¹ is —CH₂N(H)R¹⁵; R² is —OH; R³, R⁴, R⁵, and R⁶ are hydrogen; R⁷ is hydrogen or —C(O)R¹⁰; R¹⁰ is alkyl; and R¹⁵ is alkyl substituted with a substituent selected from the group consisting of phenyl and pyridyl, in which the phenyl is unsubstituted or substituted with 1,2,3-thiadiazolyl;

compounds having formula (I), and salts, prodrugs, and salts of prodrugs thereof, in which R¹ is (((4-(1,2,3-thiadiazol-5-yl)phenyl)methyl)amino)methyl, (((phenyl)methyl)amino)methyl, ((2-(phenyl)ethyl)amino)methyl, ((3-(phenyl)propyl)amino)methyl, or ((pyridin-3-yl)methyl)aminomethyl, R² is —OH; and R³, R⁴, R⁵, R⁶, and R⁷ are hydrogen;

compounds having formula (I), and salts, prodrugs, and salts of prodrugs thereof, in which R¹ is (((4-(1,2,3-thiadiazol-5-yl)phenyl)methyl)amino)methyl, (((phenyl)methyl)amino)methyl, ((2-(phenyl)ethyl)amino)methyl, ((3-(phenyl)propyl)amino)methyl, or ((pyridin-3-yl)methyl)aminomethyl; R² is —OH; R³, R⁴, R⁵, and R⁶ are hydrogen; and R⁷ is —C(O)((2-methyl)propyl);

compounds and salts, prodrugs, and salts of prodrugs thereof, having formula (II)

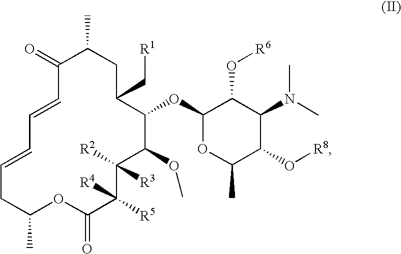

in which R¹ is —CH₂N(H)R¹⁵; R² is —OH; R³, R⁴, R⁵, R⁶, and R⁸ are hydrogen; and R¹⁵ is alkyl substituted with phenyl, in which the phenyl is substituted with 1,2,3-thiadiazolyl;

compounds having formula (II), and salts, prodrugs, and salts of prodrugs thereof, in which R¹ is (((4-(1,2,3-thiadiazol-5-yl)phenyl)methyl)amino)methyl; R² is —OH; and R³, R⁴, R⁵, R⁶, and R⁸ are hydrogen; and

compounds, and salts, prodrugs, and salts of prodrugs thereof, which are

(4R,5S,6S,7R,9R,16R)-6-(((2S,3R,4S,5S,6R)-4-(dimethylamino)-3,5-dihydroxy-6-methyltetrahydro-2H-pyran-2-yl)oxy)-4-hydroxy-5-methoxy-9,16-dimethyl-7-(2-((4-(1,2,3-thiadiazol-5-yl)benzyl)amino)ethyl)oxacyclohexadeca-11,13-diene-2,10-dione;

(4R,5S,6S,7R,9R,16R)-6-(((2S,3R,4R,5S,6R)-5-(((2S,4R,5S,6S)-4,5-dihydroxy-4,6-dimethyltetrahydro-2H-pyran-2-yl)oxy)-4-(dimethylamino)-3-hydroxy-6-methyltetrahydro-2H-pyran-2-yl)oxy)-4-hydroxy-5-methoxy-9,16-dimethyl-7-(2-((4-(1,2,3-thiadiazol-5-yl)benzyl)-amino)ethyl)oxacyclohexadeca-11,13-diene-2,10-dione;

(2S,3S,4R,6S)-6-(((2R,3S,4R,5R,6S)-4-(dimethylamino)-5-hydroxy-6-(((4R,5S,6S,7R,9R,16R)-4-hydroxy-5-methoxy-9,16-dimethyl-2,10-dioxo-7-(2-((4-(1,2,3-thiadiazol-5-yl)benzyl)amino)ethyl)oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate;

(2S,3S,4R,6S)-6-(((2R,3S,4R,5R,6S)-6-(((4R,5S,6S,7R,9R,16R)-7-(2-(benzylamino)ethyl)-4-hydroxy-5-methoxy-9,16-dimethyl-2,10-dioxooxacyclohexadeca-11,13-dien-6-yl)oxy)-4-(dimethylamino)-5-hydroxy-2-methyltetrahydro-2H-pyran-3-yl)oxy)-4-hydroxy-2,4-dimethyltetrahydro-2H-pyran-3-yl 3-methylbutanoate;

(2S,3S,4R,6S)-6-(((2R,3S,4R,5R,6S)-4-(dimethylamino)-5-hydroxy-6-(((4R,5S,6S,7R,9R,16R)-4-hydroxy-5-methoxy-9,16-dimethyl-2,10-dioxo-7-(2-((2-phenylethyl)amino)ethyl)-oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate;

(2S,3S,4R,6S)-6-(((2R,3S,4R,5R,6S)-4-(dimethylamino)-5-hydroxy-6-(((4R,5S,6S,7R,9R,16R)-4-hydroxy-5-methoxy-9,16-dimethyl-2,10-dioxo-7-(2-((3-phenylpropyl)amino)ethyl)-oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate;

(2S,3S,4R,6S)-6-(((2R,3S,4R,5R,6S)-4-(dimethylamino)-5-hydroxy-6-(((4R,5S,6S,7R,9R,16R)-4-hydroxy-5-methoxy-9,16-dimethyl-2,10-dioxo-7-(2-((pyridin-3-ylmethyl)amino)ethyl)-oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate.

Compounds of this invention contain asymmetrically substituted carbon atoms in the R or S configuration, in which the terms “R” and “S” are as defined by the IUPAC 1974 Recommendations for Section E, Fundamental Stereochemistry, Pure Appl. Chem. (1976) 45, 13-10. Compounds having asymmetrically substituted carbon atoms with equal amounts of R and S configurations are racemic at those carbon atoms. Atoms with an excess of one configuration over the other are assigned the configuration which is present in the higher amount, preferably an excess of about 85%-90%, more preferably an excess of about 95%-99%, and still more preferably an excess greater than about 99%. Accordingly, this invention is meant to embrace all stereoisomers of the compounds including racemic mixtures, enantiomers, mixtures of enantiomers, diastereomers, and mixtures of diastereomers.

Individual stereoisomers of the compounds may be prepared by any one of a number of methods within the knowledge of the ordinarily skilled practioner. These methods include stereospecific synthesis, chromatographic separation of diastereomers, chromatographic resolution of enantiomers, enzymatic resolution, and conversion of enantiomers in an enantiomeric mixture to diastereomers and chromatographically separating the diastereomers and regeneration of the individual enantiomers.

Stereospecific synthesis involves the use of appropriate chiral starting materials and synthetic reactions which do not cause racemization or inversion of stereochemistry at the chiral centers.

Diastereomeric mixtures of compounds resulting from a synthetic reaction can be separated by chromatographic techniques which are well-known to the ordinarily skilled practioner.

Chromatographic resolution of enantiomers can be accomplished on chiral commercially available chromatography resins. In practice, the racemate is placed in solution and loaded onto the column containing a chiral stationary phase. The enantiomers are then separated by high performance liquid chromatography.

Enzymes, such as esterases, phosphatases and lipases, may be useful for resolution of derivatives of the enantiomers in an enantiomeric mixture. For example, an ester derivative of a carboxyl group of the compounds to be separated can be prepared. Certain enzymes will selectively hydrolyze only one of the enantiomers in the mixture. Then the resulting enantiomerically pure acid can be separated from the unhydrolyzed ester.

Resolution of enantiomers may also be accomplished by converting the enantiomers in the mixture to diastereomers by reacting of the former and chiral auxiliaries. The resulting diastereomers can then be separated by column chromatography. This technique is especially useful when the compounds to be separated contain a carboxyl, amino or hydroxyl group that will form a salt or covalent bond with the chiral auxiliary. Chirally pure amino acids, organic carboxylic acids or organosulfonic acids are especially useful as chiral auxiliaries. Once the diastereomers have been separated by chromatography, the individual enantiomers can be regenerated. Frequently, the chiral auxiliary can be recovered and reused.

Compounds of this invention may also contain carbon-carbon double bonds or carbon-nitrogen double bonds in the Z or E configuration, in which the term “Z” represents the larger two substituents on same side of a carbon-carbon or carbon-nitrogen double bond and the term “E” represents the larger two substituents on opposite sides of a carbon-carbon or carbon-nitrogen double bond. The compounds may also exist as an equilibrium mixture of Z or E configurations.

Compounds of this invention which contain —OH, —NH—, or —CO₂H moieties may have attached thereto prodrug-forming moieties. The prodrug-forming moieties are removed by metabolic processes and release the compounds having the freed hydroxyl, amino, or carboxylic acid in vivo. Prodrugs are useful for adjusting such pharmacokinetic properties of the compounds as solubility and/or hydrophobicity, absorption in the gastrointestinal tract, bioavailability, tissue penetration, and rate of clearance.

Compounds of this invention may exist as acid addition salts, basic addition salts, or zwitterions. Salts of the compounds are prepared during their isolation or following their purification. Acid addition salts of the compounds are those derived from the reaction of the compounds with an acid. For example, the acetate, adipate, alginate, bicarbonate, citrate, aspartate, benzoate, benzenesulfonate, bisulfate, butyrate, camphorate, camphorsufonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate, and undecanoate salts of the compounds and prodrugs thereof are contemplated as being embraced by this invention. When the compounds contain —CO₂H moieties, basic addition salts may be prepared therefrom by reaction with a base such as the hydroxide, carbonate, and bicarbonate of cations such as lithium, sodium, potassium, calcium, and magnesium.

Compounds of this invention may be administered with or without an excipient. Excipients include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof. Excipients for orally administered compounds in solid dosage forms include agar, alginic acid, aluminum hydroxide, benzyl alcohol, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, Ringer's solution, talc, tragacanth, water, safflower oil, sesame oil, sodium carboxymethyl cellulose, sodium lauryl sulfate, sodiumphosphate salts, soybean oil, sucrose, tetrahydrofurfuryl alcohol, and mixtures thereof. Excipients for ophthalmically and orally administered compounds in liquid dosage forms include benzyl alcohol, benzyl benzoate, 1,3-butylene glycol, castor oil, corn oil, cottonseed oil, ethanol, ethyl acetate, ethyl carbonate, fatty acid esters of sorbitan, germ oil, groundnut oil, glycerol, isopropanol, olive oil, polyethylene glycols, propylene glycol, sesame oil, tetrahydrofurfuryl alcohol, water, and mixtures thereof. Excipients for osmotically administered compounds include chlorofluorohydrocarbons, ethanol, isopropanol, water, and mixtures thereof. Excipients for parenterally administered compounds include 1,3-butanediol, castor oil, corn oil, cottonseed oil, germ oil, groundnut oil, liposomes, oleic acid, olive oil, peanut oil, Ringer's solution, safflower oil, sesame oil, soybean oil, U.S.P. or isotonic sodium chloride solution, water, and mixtures thereof. Excipients for rectally and vaginally administered compounds include cocoa butter, polyethylene glycol, wax, and mixtures thereof.

Compounds of this invention may be administered orally, ophthalmically, osmotically, parenterally (subcutaneously, intramuscularly, intrasternally, intravenously), rectally, topically, transdermally, and vaginally. Orally administered compounds in solid dosage forms may be administered as capsules, dragees, granules, pills, powders, and tablets. Ophthalmically and orally administered compounds in liquid dosage forms may be administered as elixirs, emulsions, microemulsions, solutions, suspensions, and syrups. Osmotically and topically administered compounds may be administered as creams, gels, inhalants, lotions, ointments, pastes, powders, solutions, and sprays. Parenterally administered compounds may be administered as aqueous or oleaginous solutions or aqueous or oleaginous suspensions, in which suspensions comprise crystalline, amorphous, or otherwise insoluble forms of the compounds. Rectally and vaginally administered compounds may be administered as creams, gels, lotions, ointments, and pastes.

Therapeutically effective amounts of compounds of this invention depend on the species being treated, the disorder being treated and the severity thereof, the composition comprising the compounds, the time of administration, the route of administration, the duration of treatment, the potency of the compounds, and the rate of excretion of the compounds. The daily therapeutically effective amount of the compounds administered to a patient in single or divided doses range from about 0.1 to about 200 mg/kg body weight, preferably from about 0.25 to about 100 mg/kg body weight. Single dose compositions contain these amounts of the compounds or combinations of submultiples thereof.

To determine antibacterial activity of compounds of this invention, twelve petri dishes, each containing successive aqueous dilutions of test compounds in sterilized Brain Heart Infusion agar (Difco 0418-01-5) (10 mL), were inoculated with 1:100 dilutions of the representative microorganisms in TABLE 1 using a Steers replicator block (or 1:10 dilutions for slow-growing Streptococcus strains), co-incubated at 35-37° C. for 20-24 hours with a control plate having no compound, and inspected visually to provide the minimum inhibitory concentration (MIC), in μg/mL, by which is meant the lowest concentration of the test compound which yielded no growth, a slight haze, or sparsely isolated colonies on the inoculums spot as compared to growth in the control plate.

TABLE 1

| Microorganism | Code |
|---|---|
| Staphylococcus aureus NCTC10649M | AA |
| Staphylococcus aureus A5177 | BB |
| Staphylococcus aureus PIU 2043 | CC |
| Staphylococcus aureus 1775 | DD |
| Streptococcus pyrogenes EES61 | EE |
| Streptococcus pyrogenes 930 | FE |
| Streptococcus pyrogenes PIU 2548 | GC |
| Streptococcus pneumoniae ATCC 6303 | HH |
| Streptococcus pneumoniae 5979 | JJ |
| Streptococcus pneumoniae 5649 | KK |
| Enterococcus faecalis PIU 1967 | LL |
| Enterococcus faecium GYR 1632 | MM |
| Moraxella catarrhalis 2604 | NN |
| Haemophilus influenzae GYR 1435 | PP |
| Escherichia coli JUHL | QQ |

Compounds of this invention displayed antibacterial activity superior to the control and in the range of about 1 μg/mL to greater than about 128 μg/mL against the microorganisms listed in TABLE 1, which control demonstrated no antibacterial activity. This antibacterial activity demonstrates the usefulness of the compounds as antibacterials.

It is also meant to be understood that certain metabolites of compounds of this invention, which metabolites are produced by in vitro or in vivo metabolic processes, would also be useful as antibacterials and are meant to be embraced by this invention.

It is still also meant to be understood that certain precursor compounds, which precursor compounds may be metabolized in vitro or in vivo to form compounds of this invention, would also be useful as antibacterials and are meant to be embraced by this invention.

Compounds of this invention may be prepared by in vitro or in vivo metabolic processes or by synthetic chemical processes, examples of which synthetic chemical processes, and intermediates used in the processes, are shown hereinbelow. It is meant to be understood that the order of the steps in the processes may be varied, equivalent reagents, solvents, and reaction conditions may be substituted for those specifically mentioned, and vulnerable moieties may be protected and deprotected during the process.

Abbreviations used herein are DMF for N,N-dimethylformamide, DME from 1,2-dimethoxyethane, and THF for tetrahydrofuran.

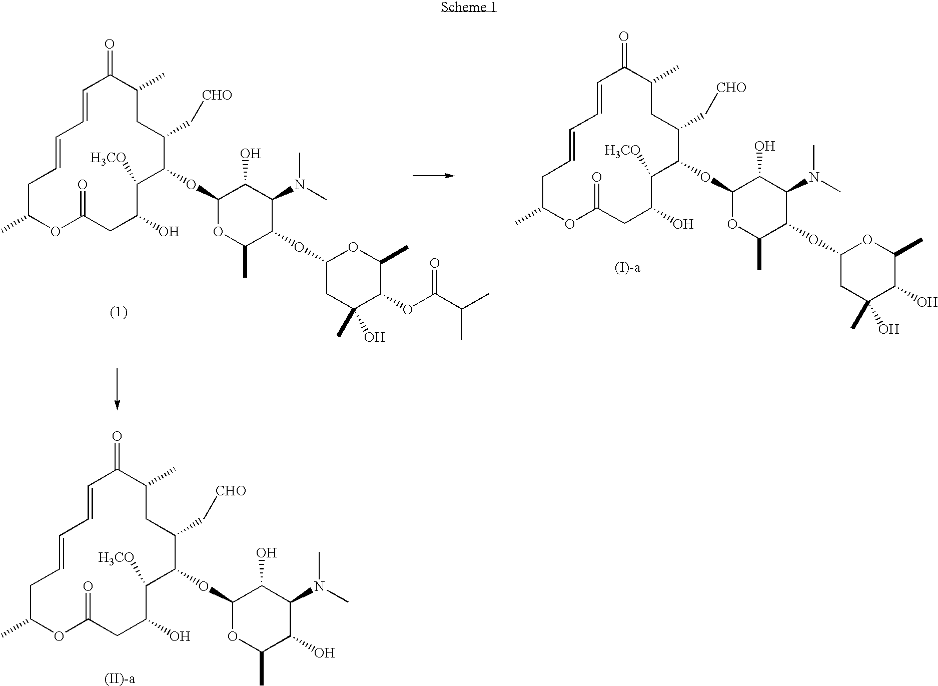

Niddamycin (1) may be converted to 4″-O-desisovaleryl niddamycin (compounds having formula (I)-a) by reacting the former and a first base. First bases include lithium, hydroxide, sodium hydroxide, and potassium hydroxide. The reaction is typically conducted from about 1 to about 24 hours, at about 0° C. to about 25° C., in mixtures of solvents comprising water and one or more of ethyl acetate, iso-propyl acetate, THF, 1,4-dioxane, and DME.

Niddamycin may also be converted to desmycarosyl niddamycin (compounds having formula (II)-a) by reacting the former and a first acid. First acids include hydrochloric acid, and hydrobromic acid. The reaction is typically conducted from about 1 to 24 hours, at about 0° C. to 25° C., in mixtures of solvents comprising water and one or more of ethyl acetete, iso-propyl acetate, THF, 1,4-dioxane, and DME.

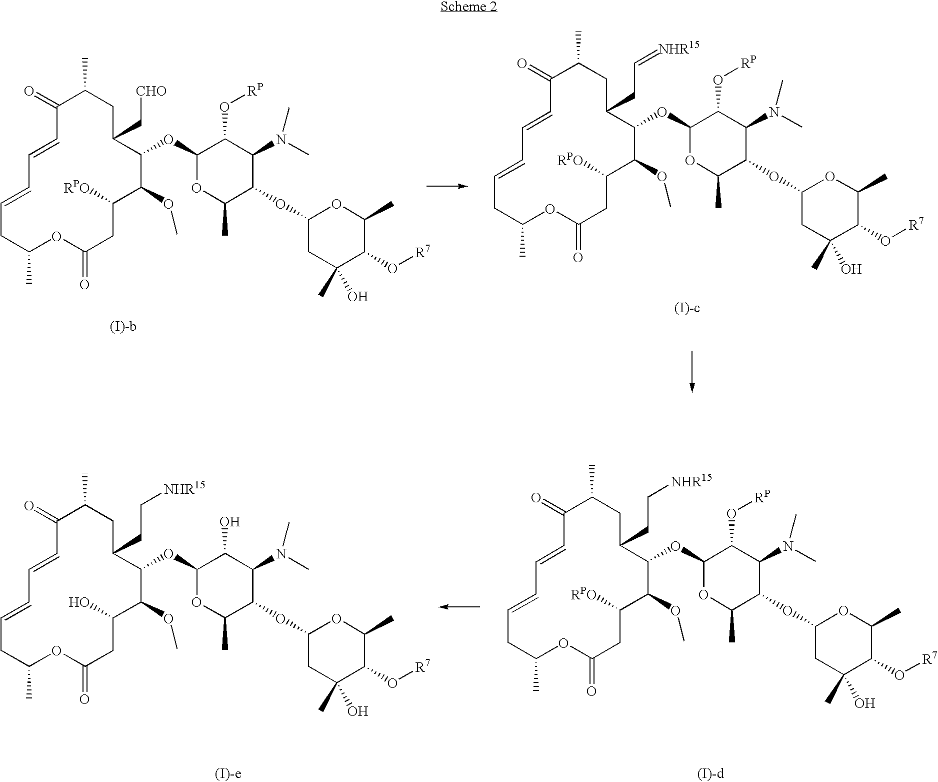

Compounds having formula (I)-b in which R^{P} is acyl, benzoyl or trialkylsilyl, may be converted to compounds having formula (I)-c by reacting the former with an amine having formula NH₂R¹⁵ and a second acid.

Examples of amines having formula NH₂R¹⁵ include dimethylamine, ethylamine, (prop-2-enyl)amine, (prop-2-ynyl)amine, (phenyl)methylamine, (quinolin-3-ylmethyl)amine, (isoquinolin-3-ylmethyl)amine, (phenylethyl) amine, (phenylpropyl) amine, ((pyridin-3-yl)methyl)amine; ((5-pyridin-2-yl)thien-2-yl)methyl)amine, (((3-pyridin-2-yl)isoxazol-5-yl)methyl)amine, (((5-pyrimidin-2-yl)-thien-2-yl)methyl)amine, and ((4-(1,2,3-thiadiazol-5-yl)phenyl)methyl)amine.

Examples of second acids include hydrochloric acid, para-toluenesulfonic acid, acetic acid, formic acid, boron trifluoride, and aluminum chloride.

The reaction is typically conducted at about −10° C. to about 50° C. over about 0.5 hour to about 24 hours in solvents such as dichloromethane, chloroform, tetrahydrofuran, ether, toluene, benzene, N,N-dimethylformamide, and mixtures thereof.

Compounds having formula (I)-c in which R^{P} is acetyl, benzoyl or trialkylsilyl, may be converted to compounds having formula (I)-d by reacting the former with a reducing agent.

Examples of reducing agents include hydrogen, sodium borohydride, sodium cyanoborohydride, sodium triacetoxyborohydride, zinc and hydrochloric acid, iron pentacarbonyl and alcoholic potassium hydroxide, borane-pyridine, and formic acid.

The reaction is typically conducted at about −10° C. to about 150° C., over about 1 hour to about 10 days, in solvents such as tetrahydrofuran, dichloromethane, toluene, benzene, xylene, N,N-dimethylformamide, and mixtures thereof.

Compounds having formula (I)-d in which R^{P} is acetyl or benzoyl, may be converted to compounds having formula (I)-e by reacting the former and a deprotecting agent.

Examples of deprotecting agents include acids such as methanol, ethanol, acetic acid, and formic acid and bases such as lithium hydroxide, sodium hydroxide, potassium hydroxide, potassium carbonate, and ammonia.

The reaction is typically conducted at about 25° C. to about 70° C., over about 1 hour to about 72 hours, in solvents such as water, methanol, ethanol, and mixtures thereof.

Compounds having formula (I)-d in which R^{P} is trialkylsilyl, may be converted to compounds having formula (I)-e by reacting the former and a fluoride-donating agent.

Examples of fluoride-donating agents include tetrabutylammonium fluoride, tetrabutylammonium chloride/potassium fluoride monohydrate, HF.pyridine, hydrogen fluoride, and ammonium fluoride.

The reaction is typically conducted over about 1 to about 24 hours, at about 25° C. to about 35° C., in solvents such as tetrahydrofuran, N,N-dimethylformamide, acetonitrile, methanol, ethanol, and mixtures thereof.

Other processes for making compounds of this invention are well-known in the art.

The compounds and processes of this invention will be better understood in connection with the following examples.

### EXAMPLE 1

(4R,5S,6S,7R,9R,16R)-6-(((2S,3R,4S,5S,6R)-4-(dimethylamino)-3,5-dihydroxy-6-methyltetrahydro-2H-pyran-2-yl)oxy)-4-hydroxy-5-methoxy-9,16-dimethyl-7-(2-((4-(1,2,3-thiadiazol-5-yl)benzyl)amino)ethyl)oxacyclohexadeca-11,13-diene-2,10-dione

A solution of desmycarosyl niddamycin (56 mg), 4-(1,2,3-thiadiazol-4-yl)benzylamine (38 mg), acetic acid (12 μL) and sodium sulfate (143 mg) in dichloromethane (2 mL) was stirred for 1 hour, treated with sodium triacetoxyborohydride (25 mg), stirred for 2 hours, diluted with ethyl acetate, washed with 5% Na₂CO₃, dried over anhdrous Na₂SO₄, filtered, and concentrated; and the concentrate was purified by flash chromatography on silica gel with 95:5:0.5 dichloromethane/methanol/concentrated ammonium hydroxide.

### EXAMPLE 2

(4R,5S,6S,7R,9R,16R)-6-(((2S,3R,4R,5S,6R)-5-(((2S,4R,5S,6S)-4,5-dihydroxy-4,6-dimethyltetrahydro-2H-pyran-2-yl)oxy)-4-(dimethylamino)-3-hydroxy-6-methyltetrahydro-2H-pyran-2-yl)oxy)-4-hydroxy-5-methoxy-9,16-dimethyl-7-(2-((4-(1,2,3-thiadiazol-5-yl)benzyl)-amino)ethyl)oxacyclohexadeca-11,13-diene-2,10-dione

This example was prepared by substituting 4″-O-desisovaleryl niddamycin for desmycarosyl niddamycin in EXAMPLE 1.

### EXAMPLE 3

(2S,3S,4R,6S)-6-(((2R,3S,4R,5R,6S)-4-(dimethylamino)-5-hydroxy-6-(((4R,5S,6S,7R,9R,16R)-4-hydroxy-5-methoxy-9,16-dimethyl-2,10-dioxo-7-(2-((4-(1,2,3-thiadiazol-5-yl)benzyl)amino)ethyl)oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate

This example was prepared by substituting niddamycin for desmycarosyl niddamycin in EXAMPLE 1.

### EXAMPLE 4

(2S,3S,4R,6S)-6-(((2R,3S,4R,5R,6S)-6-(((4R,5S,6S,7R,9R,16R)-7-(2-(benzylamino)ethyl)-4-hydroxy-5-methoxy-9,16-dimethyl-2,10-dioxooxacyclohexadeca-11,13-dien-6-yl)oxy)-4-(dimethylamino)-5-hydroxy-2-methyltetrahydro-2H-pyran-3-yl)oxy)-4-hydroxy-2,4-dimethyltetrahydro-2H-pyran-3-yl 3-methylbutanoate

A solution of niddamycin (157 mg) and benzylamine (43 μL) in dichloromethane (4 mL) at ambient temperature was treated with acetic acid (24 μL) and sodium sulfate (286 mg), stirred at 0° C. for 1 hour, treated with sodium triacetoxyborohydride (50 mg), stirred at ambient temperature for 2 hours, diluted with ethyl acetate, washed with 5% Na₂CO₃, dried over anhydrous Na₂SO₄, filtered, and concentrated; and the concentrate was purified by flash chromatography on silica gel with 95:5:0.5 dichloromethane/methanol/concentrated ammonium hydroxide.

### EXAMPLE 5

(2S,3S,4R,6S)-6-(((2R,3S,4R,5R,6S)-4-(dimethylamino)-5-hydroxy-6-(((4R,5S,6S,7R,9R,16R)-4-hydroxy-5-methoxy-9,16-dimethyl-2,10-dioxo-7-(2-((2-phenylethyl)amino)ethyl)-oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate

This example was prepared by substituting phenethylamine for benzylamine in EXAMPLE 4.

### EXAMPLE 6

(2S,3S,4R,6S)-6-(((2R,3S,4R,5R,6S)-4-(dimethylamino)-5-hydroxy-6-(((4R,5S,6S,7R,9R,16R)-4-hydroxy-5-methoxy-9,16-dimethyl-2,10-dioxo-7-(2-((3-phenylpropyl)amino)ethyl)-oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate

This example was prepared by substituting 3-phenyl-1-propylamine for benzylamine in EXAMPLE 4.

### EXAMPLE 7

(2S,3S,4R,6S)-6-(((2R,3S,4R,5R,6S)-4-(dimethylamino)-5-hydroxy-6-(((4R,5S,6S,7R,9R,16R)-4-hydroxy-5-methoxy-9,16-dimethyl-2,10-dioxo-7-(2-((pyridin-3-ylmethyl)amino)ethyl)-oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate

This example was prepared by substituting 3-pyridinylmethylamine for benzylamine in EXAMPLE 4.

The foregoing is merely illustrative of this invention and is not intended to limit the same. Variations, changes, and equivalents, as recited in the appended claims, are intended to be embraced by this invention.

### SPECTRAL DATA

### EXAMPLE 1

¹³C NMR (CDCl₃) δ 203.4, 172.6, 142.9, 141.4, 141.0, 131.9, 129.6, 129.5, 129.2, 127.5, 122.5, 105.1, 86.1, 78.6, 73.3, 71.3, 70.6, 70.2, 70.1, 68.6, 68.2, 61.8, 52.6, 45.5, 44.8, 41.7, 41.2, 38.4, 33.3, 33.2, 26.9, 20.4, 18.0, 17.5.

### EXAMPLE 2

¹³C NMR (CDCl₃) δ 203.3, 172.6, 142.9, 141.4, 141.0, 131.9, 129.6, 129.6, 129.2, 127.6, 127.5, 122.5, 104.6, 96.4, 86.1, 78.4, 76.4, 74.8, 73.1, 71.9, 69.3, 68.7, 68.6, 68.2, 66.0, 61.8, 52.7, 45.5, 44.8, 42.0, 40.9, 38.3, 33.3, 33.2, 25.4, 20.4, 19.2, 18.3, 17.4.

### EXAMPLE 3

¹³C NMR (CDCl₃) δ 203.3, 172.9, 172.6, 142.9, 141.5, 141.0, 131.9, 129.7, 129.5, 129.2, 127.5, 127.5, 122.5, 104.5, 96.9, 86.0, 78.4, 77.0, 75.9, 73.0, 71.9, 69.3, 68.7, 68.6, 68.2, 63.4, 61.8, 52.7, 45.5, 44.8, 43.3, 41.9, 41.7, 41.2, 38.3, 33.3, 33.2, 27.1, 25.5, 25.3, 22.4, 22.3, 20.4, 19.0, 17.8, 17.6.

### EXAMPLE 4

¹³C NMR (CDCl₃) δ 203.3, 172.9, 172.4, 142.8, 140.8, 139.7, 132.0, 128.4, 128.4, 126.9, 126.6, 104.5, 96.9, 86.0, 78.4, 77.0, 75.8, 73.0, 71.8, 69.3, 68.7, 68.5, 68.2, 63.4, 61.7, 53.0, 45.3, 44.7, 43.3, 41.9, 41.7, 41.2, 38.4, 33.2, 33.1, 27.0, 25.5, 25.3, 22.4, 22.3, 20.4, 18.9, 17.8, 17.5.

### EXAMPLE 5

¹³C NMR (CDCl₃) δ 203.1, 172.8, 172.4, 142.9, 141.0, 139.8, 131.7, 128.7, 128.4, 126.1, 122.4, 104.5, 96.9, 86.2, 78.3, 77.0, 75.8, 72.9, 71.8, 69.3, 68.6, 68.4, 68.0, 63.3, 61.7, 50.0, 45.9, 44.7, 43.2, 41.9, 41.6, 41.3, 38.3, 35.9, 33.3, 33.1, 26.9, 25.4, 25.3, 22.4, 22.3, 20.3, 18.8, 17.8, 17.5.

### EXAMPLE 6

¹³C NMR (CDCl₃) δ 203.2, 172.8, 172.3, 142.9, 142.0, 141.0, 131.8, 128.4, 128.2, 125.6, 122.3, 104.5, 96.9, 86.1, 78.4, 77.0, 75.7, 72.9, 71.8, 69.3, 68.6, 68.4, 68.0, 63.3, 61.7, 48.4, 45.9, 44.7, 43.2, 41.9, 41.6, 41.1, 38.3, 33.5, 33.3, 33.1, 31.3, 27.0, 25.4, 25.3, 22.4, 22.3, 20.3, 18.9, 17.8, 17.5.

### EXAMPLE 7

¹³C NMR (CDCl₃) δ 203.2, 172.9, 172.8, 149.7, 148.4, 142.9, 141.0, 136.1, 135.3, 131.8, 123.4, 122.4, 104.4, 96.9, 85.9, 78.2, 77.0, 75.8, 73.0, 71.8, 69.3, 68.7, 68.6, 68.1, 63.4, 61.7, 50.5, 45.7, 44.8, 43.3, 41.9, 41.7, 41.3, 38.2, 33.3, 33.2, 31.5, 27.2, 25.5, 25.3, 22.4, 22.3, 20.3, 18.9, 17.8, 17.4.

The foregoing is merely illustrative of the invention and is not intended to limit the same to the disclosed compounds and proceses. Variations and changes which are obvious to one skilled in the art are intended to be within the scope and nature of the invention as defined in the claims.

What is claimed is:

1. A compound, or salt, prodrug, or salt of a prodrug thereof, having formula (I) 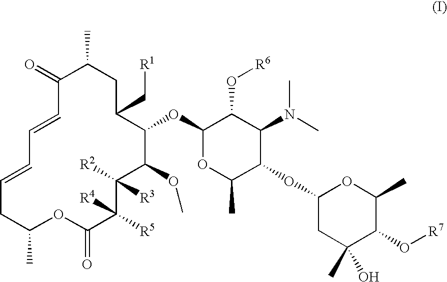
or formula (II) 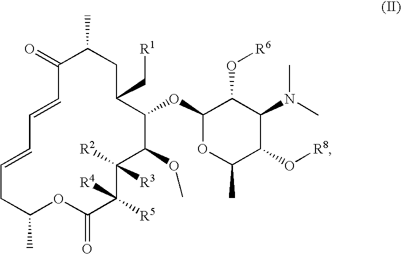
in which
R¹ is hydrogen, alkyl, cycloalkyl, alkenyl, alkynyl, —R⁹, —C (O) H, —C (O) OH, —C (O) R¹⁰, —C (O) NR¹¹R¹², —C(H)═N—OR¹³, —C(H)═NR¹⁴, —CH₂N(H)R¹⁵, or —CH₂NR¹⁵R¹⁶;
one of R² or R³ is hydrogen and the other is —OH, —OCH₂R⁹, —OC(O)R¹⁰, —OC (O)NR¹¹R¹², —OCH₂C (H) ═N—OR¹³, or —OCH₂C(H)═NR¹⁴; or
R² and R³ together are ═O;
one of R⁴ or R⁵ is hydrogen and the other is hydrogen or fluoro;
R⁶, R⁷, and R⁸ are independently hydrogen, alkyl, cycloalkyl, —(CH₂)alkenyl, —(CH₂)alkynyl, —R^{P}, —CH₂R⁹, —C(O)R¹⁰, —C(O)NR¹¹R¹², —(CH₂)C(H)═N—OR¹³, —(CH₂)₂C(H)═NR¹⁴, —(CH₂)₂N(H)R¹⁵, —(CH₂)₂NR¹⁵R¹⁶, alkyl substituted with one substituent selected from the group consisting of aryl, heteroaryl, and heterocyclyl, —(CH₂)alkenyl substituted with one substituent selected from the group consisting of —NR¹⁵R¹⁶, aryl, heteroaryl, and heterocyclyl, or —(CH₂)alkynyl substituted with one substituent selected from the group consisting of —NR¹⁵R¹⁶, aryl, heteroaryl, and heterocyclyl;
R^{P} is a hydroxyl protecting moiety;
R⁹ is alkyl interrupted with one or two moieties independently selected from the group consisting of —O—, —S—, —S(O)—, and —SO₂—, alkenyl interrupted with one moiety selected from the group consisting of —O—, —S—, —S(O)—, and —SO₂—, or alkynyl interrupted with one moiety selected from the group consisting of —O—, —S—, —S(O)—, and —SO₂—,
in which each R⁹ moiety is unsubstituted or substituted with one, two, or three substituents independently selected from the group consisting of cycloalkyl, halo, aryl, heteroaryl, heterocyclyl, —OH, ═O, —NH₂, —NH(alkyl), and —N(alkyl)₂;
R¹⁰ is alkyl, alkenyl, alkynyl, cycloalkyl, —R¹⁷, alkyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, alkenyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, or alkynyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂; R¹¹, R¹², R¹³, R¹⁴, and R¹⁵ are independently alkyl, —(CH₂)alkenyl, —(CH₂)alkynyl, cycloalkyl, —CH₂R¹⁷, alkyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, alkenyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, or alkynyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂;
R¹⁶ is hydrogen or R¹⁵ ; and
R¹⁷ is alkyl interrupted with one or two moieties independently selected from the group consisting of —O—, —S—, —S(O)—, and —SO₂—, alkenyl interrupted with one moiety selected from the group consisting of —O—, —S—, —S(O)—, and —SO₂—, or alkynyl interrupted with one moiety selected from the group consisting of —O—, —S—, —S(O)—, and —SO₂—,
in which each R¹⁷ moiety is unsubstituted or substituted with one, two, or three substituents independently selected from the group consisting of cycloalkyl, halo, aryl, heteroaryl, heterocyclyl, —OH, ═O, —NH₂, —NH(alkyl), and —N(alkyl)₂—.

2. A compound of claim 1, or salt, prodrug, or salt of a prodrug thereof, having formula (I) or formula (II), in which
R¹ is hydrogen, alkyl, cycloalkyl, alkenyl, alkynyl, —C(O)H, —C(O)OH, —C(O)R¹⁰, —C(O)NR¹¹R¹², —C(H)═N—OR¹³, —C(H)═NR¹⁴, —CH₂N(H)R¹⁵, or —CH₂NR¹⁵R¹⁶;
one of R² or R³ is hydrogen and the other is —OH; or
R² and R³ together are ═O;
one of R⁴ or R⁵ is hydrogen and the other is hydrogen or fluoro;
R⁶ is hydrogen or —R^{P};
R⁷ and R⁸ are independently hydrogen, —R^{P} or —C(O)R¹⁰;
R^{P} is a hydroxyl protecting moiety;
R¹⁰ is alkyl, alkenyl, alkynyl, cycloalkyl, alkyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, alkenyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, or alkynyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂;
R¹¹, R¹², R¹³, R¹⁴, and R¹⁵ are independently alkyl, —(CH₂)alkenyl, —(CH₂)alkynyl, cycloalkyl, alkyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, alkenyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, or alkynyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂; and
R¹⁶ is hydrogen or R¹⁵.

3. A compound of claim 2 or salt, prodrug, or salt of a prodrug thereof, having formula (I) or formula (II), in which
compounds having formula (I) and formula (II), and salts, prodrugs, and salts of prodrugs thereof, in which
R¹ is —C (H) ═NR¹⁴, —CH₂N (H)R¹⁵, or —CH₂NR¹⁵R¹⁶;
one of R² or R³ is hydrogen and the other is —OH; or
R² and R³ together are ═O;
one of R⁴ or R⁵ is hydrogen and the other is hydrogen or fluoro;
R⁶ is hydrogen or —R^{P};
R⁷, and R⁸ are independently hydrogen, —R^{P} or —C(O) (alkyl);
R^{P} is a hydroxyl protecting moiety; and
R¹⁴ and R¹⁵ are independently alkyl, —(CH₂)alkenyl, —(CH₂)alkynyl, cycloalkyl, alkyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, alkenyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂, or alkynyl substituted with one, two, or three substituents independently selected from the group consisting of cycloalkyl, halo, aryl, heteroaryl, heterocyclyl, —OH, ═O, —NH₂, —NH(alkyl), and —N(alkyl)₂; and
R¹⁶ is hydrogen or R¹⁵.

4. A compound of claim 3 or salt, prodrug, or salt of a prodrug thereof, having formula (I) or formula (II), in which
R¹ is —C(H)═NR¹⁴, —CH₂N(H)R¹⁵, or —CH₂NR¹⁵R¹⁶;
one of R² or R³ is hydrogen and the other is —OH; or
R² and R³ together are ═O;
one of R⁴ or R⁵ is hydrogen and the other is hydrogen or fluoro;
R⁶ is hydrogen or —R^{P};
R⁷ and R⁸ are independently hydrogen, —R^{P} or —C(O) (alkyl);
R^{P} is a hydroxyl protecting moiety;
R¹⁴and R¹⁵ are independently alkyl, —(CH₂)alkenyl, —(CH₂)alkynyl, cycloalkyl, or alkyl substituted with one substituent selected from the group consisting of phenyl, pyridyl, pyridmidyl, thienyl, imidazolyl, oxazolyl, isoxazolyl, thiazolyl, oxadiazolyl, thiadiazolyl and triazolyl, each of which is independently unfused or fused with a moiety selected from the group consisting of phenyl, pyridyl, and pyrimidyl and independently unsubstituted or substituted with one substituent selected from the group selected from phenyl, pyridyl, pyridmidyl, thienyl, imidazolyl, oxazolyl, isoxazolyl, thiazolyl, oxadiazolyl, thiadiazolyl and triazolyl; and
R¹⁶ is hydrogen or R¹⁵.

5. A compound of claim 4 or salt, prodrug, or salt of a prodrug thereof, having formula (I) or formula (II), in which
R¹ is (prop—2-enyl)aminomethyl, (prop-2-ynyl)aminomethyl, (((phenyl)methyl)amino)methyl, (quinolin-3-ylmethyl)aminomethyl, (isoquinolin-3-ylmethyl)aminomethyl, ((2-(phenyl)ethyl)amino)methyl, ((3-(phenyl)propyl)amino)methyl, ((pyridin-3-yl)methyl)aminomethyl; ((5-pyridin-2-yl)thien-2-yl)methyl)aminomethyl, (((3-pyridin-2-yl)isoxazol-5-yl)methyl)aminomethyl, (((5-pyrimidin-2-yl)thien-2-yl)methyl)aminomethyl, or (((4-(1,2,3-thiadiazol-5-yl)phenyl)methyl)amino)methyl;
one of R² or R³ is hydrogen and the other is —OH; or
R² and R³ together are ═O;
one of R⁴ or R⁵ is hydrogen and the other is hydrogen or fluoro;
R⁶ is hydrogen; and
R⁷ and R⁸ are independently hydrogen, —C(O)(methyl), —C(O)(ethyl), —C(O)(propyl), or —C(O)((2-methyl)propyl).

6. A compound of claim 1, or a salt, prodrug, or a salt of a prodrug thereof, having formula (I) 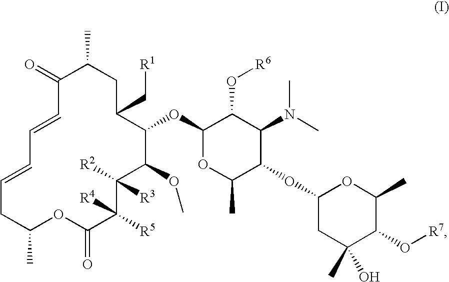
in which R¹ is —CH₂N (H)R¹⁵; R² is —OH; R³, R⁴, R⁵, and R⁶ are hydrogen; R⁷ is hydrogen or —C(O)R¹⁰; R¹⁰ is alkyl; and R¹⁵ is alkyl substituted with a substituent selected from the group consisting of phenyl and pyridyl, in which the phenyl is unsubstituted or substituted with 1,2,3-thiadiazolyl.

7. A compound of claim 6, or a salt, prodrug, or a salt of a prodrug thereof, having formula (I), in which R¹ is (((4-(1,2,3-thiadiazol-5-yl)phenyl)methyl)amino)methyl, (((phenyl)methyl)amino)methyl, ((2-(phenyl)ethyl)amino)methyl, ((3-(phenyl)propyl)amino)methyl, or ((pyridin-3-yl)methyl)aminomethyl, R² is —OH; and R³, R⁴, R⁵, R⁶, and R⁷ are hydrogen.

8. A compound of claim 6, or a salt, prodrug, or a salt of a prodrug thereof, having formula (I), in which R¹ is (((4-(1,2,3-thiadiazol-5-yl)phenyl)methyl)amino)methyl, (((phenyl)methyl)amino)methyl, ((2-(phenyl)ethyl)amino)methyl, ((3-(phenyl)propyl)amino)methyl, or ((pyridin-3-yl)methyl)aminomethyl; R² is —OH; R³, R⁴, R⁵, and R⁶ are hydrogen; and R⁷ is —C(O) (2-(methyl)propyl).

9. A compound of claim 1, or a salt, prodrug, or a salt of a prodrug thereof, having formula (II) 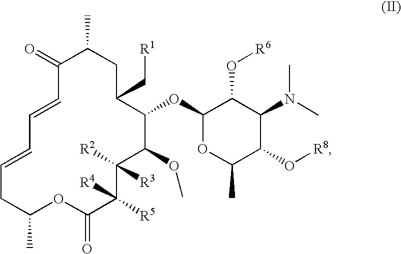
in which R¹ is —CH₂N(H)R¹⁵; R² is —OH; R³, R⁴, R⁵, R⁶, and R⁸ are hydrogen; and R¹⁵ is alkyl substituted with phenyl, in which the phenyl is substituted with 1,2,3-thiadiazolyl.

10. A compound of claim 9, or a salt, prodrug, or a salt of a prodrug thereof, having formula (II), in which R¹ is (((4-(1,2,3-thiadiazol-5-yl)phenyl)methyl)amino)methyl; R² is —OH; and R³, R⁴, R⁵, R⁶, and R⁸ are hydrogen.

11. A composition for prophylaxis or treatment of bacterial infections in a fish or a mammal comprising a therapeutically effective amount of a compound of claim 1.

12. A method for prophylaxis or treatment of bacterial infections in a fish or a mammal comprising administering to the fish or the mammal a therapeutically effective amount of a compound of claim 1.

13. A compound, or a salt, prodrug, or a salt of a prodrug thereof, which is
(4S,5S,6S,7R,9R,16R)-6-(((2S,3R,4S,5S,6R)-4-(dimethylamino)-3,5-dihydroxy-6-methyltetrahydro-2H-pyran-2-yl)oxy)-4-hydroxy-5-methoxy-9,16-dimethyl-7-(2-((4-(1,2,3-thiadiazol-5-yl)benzyl)amino)ethyl)oxacyclohexadeca-11,13-diene-2,10-dione;
(4S,5S,6S,7R,9R,16R)-6-(((2S,3R,4R,5S,6R)-5-(((2S,4R,5S,6S)-4,5-dihydroxy-4,6-dimethyltetrahydro-2H-pyran-2-yl)oxy)-4-(dimethylamino)-3-hydroxy-6-methyltetrahydro-2H-pyran-2-yl)oxy)-4-hydroxy-5-methoxy-9,16-dimethyl-7-(2-((4-(1,2,3-thiadiazol-5-yl)benzyl)amino)ethyl)oxacyclohexadeca-11,13-diene-2,10-dione;
(2S,3S,4R,6S)-6-(((2R,3S,4R,5R,6S)-4-(dimethylamino)-5-hydroxy-6-(((4S,5S,6S,7R,9R,16R)-4-hydroxy-5-methoxy-9,16-dimethyl-2,10-dioxo-7-(2-((4-(1,2,3-thiadiazol-5-yl)benzyl)amino)ethyl)oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate;
(2S,3S,4R,6S)-6-(((2R,3S,4R,5R,6S)-6-(((4S,5S,6S,7R,9R,16R)-7-(2-(benzylamino)ethyl)-4-hydroxy-5-methoxy-9,16-dimethyl-2,10-dioxooxacyclohexadeca-11,13-dien-6-yl)oxy)-4-(dimethylamino)-5-hydroxy-2-methyltetrahydro-2H-pyran-3-yl)oxy)-4-hydroxy-2,4-dimethyltetrahydro-2H-pyran-3-yl 3-methylbutanoate;
(2S,3S,4R,6S)-6-(((2R,3S,4R,5R,6S)-4-(dimethylamino)-5-hydroxy-6-(((4S,5S,6S,7R,9R,16R)-4-hydroxy-5-methoxy-9,16-dimethyl-2,10-dioxo-7-(2-((2-phenylethyl)amino)ethyl)oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate;
(2S,3S,4R,6S)-6-(((2R,3S,4R,5R,6S)-4-(dimethylamino)-5-hydroxy-6-(((4S,5S,6S,7R,9R,16R)-4-hydroxy-5-methoxy-9,16-dimethyl-2,10-dioxo-7-(2-((3-phenylpropyl)amino)ethyl)oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate; or
(2S,3S,4R,6S)-6-(((2R,3S,4R,5R,6S)-4-(dimethylamino)-5-hydroxy-6-(((4S,5S,6S,7R,9R,16R)-4-hydroxy-5-methoxy-9,16-dimethyl-2,10-dioxo-7-(2-((pyridin-3-ylmethyl)amino)ethyl)oxacyclohexadeca-11,13-dien-6-yl)oxy)-2-methyltetrahydro-2H-pyran-3-yl)oxy)-4-hydroxy-2,4-dimethyltetrahydro-2H-pyran-3-yl 3-methylbutanoate.
